(19) United States
(12) Patent Application Publication
(10) Pub. No.: US 2025/0064314 A1
(43) Pub. Date: Feb. 27, 2025

(54) AUTOREFRACTIVE DEVICE

(71) Applicant: REMIDIO INNOVATIVE SOLUTIONS PVT. LTD, Bangalore, Karnataka (IN)

(72) Inventors: Shanmuganathan NAGARAJAN, Thiruvarur (IN); Anand SIVARAMAN, Karnataka (IN)

(21) Appl. No.: 18/727,619

(22) PCT Filed: Jan. 10, 2023
(86) PCT No.: PCT/IN2023/050023

§ 371 Date: Jul. 9, 2024

(30) Foreign Application Priority Data
Jan. 10, 2022 (IN) 202241001275

(51) Int. Cl. A61B 3/028; A61B 3/00; A61B 3/12

(52) U.S. Cl. CPC A61B 3/028; A61B 3/0033; A61B 3/0041; A61B 3/1208

(57) ABSTRACT

Methods and devices for detecting refraction parameter involve an initial pattern displayed on a screen of a first detection unit of a handheld portable autorefractive device for a user to view through a viewing unit. In response to the initial pattern displayed on the screen, the refractive parameter and visual acuity of the eye of the user are detected based on feedback provided by the user or may be detected automatically by the autorefractive device. The refractive parameter value and vision acuity value detected are displayed on a display screen of the autorefractive device.

### TECHNICAL FIELD

The present subject matter relates, in general, to an autorefractive device. In particular, the present subject matter relates to a handheld portable autorefractive device.

### BACKGROUND

Autorefractive devices are optical instruments commonly used in refractive parameter correction and visual acuity determination. Techniques such as objective refraction and subjective refraction help in determining the refractive parameter of an eye, and visual acuity techniques help in determining an ability of an eye to identify objects placed at a predefined distance. Objective refraction techniques of determining the refractive parameter of a subject taking the test, are independent of an input from the subject. Whereas subjective refraction techniques involve determining the refractive parameter of a subject based on a feedback provided by the subject. Autorefractive devices are generally used to determine the refractive parameter of the eye in the form of a spherical aberration component or a cylindrical aberration component along an axis to correct the refractive parameter.

### BRIEF DESCRIPTION OF DRAWINGS

The features, aspects, and advantages of the present subject matter will be better understood with regard to the following description and accompanying figures. The use of the same reference number in different figures indicates similar or identical features and components.

FIG. 1 illustrates a block diagram of a handheld portable autorefractive device, in accordance with an example of the present subject matter.

FIG. 2 illustrates a perspective view of a first example implementation of the handheld portable autorefractive device, in accordance with an example of the present subject matter.

FIG. 3 illustrates an exploded view of the first example implementation of the handheld portable autorefractive device, in accordance with an example of the present subject matter.

FIG. 4(a) illustrates a sectional view of the first example implementation of the handheld portable autorefractive device, in accordance with an example of the present subject matter.

FIG. 4(b) illustrates a ray diagram depicting an eye of the user and an arrangement of the obstacle, the objective lens, the relay lens, and the screen to display a refractive parameter value of +15 diopter, in accordance with an example of the present subject matter.

FIG. 4(c) illustrates a ray diagram depicting an eye of the user and an arrangement of the obstacle, the objective lens, the relay lens, and the screen to display a refractive parameter value of 0 diopter, in accordance with an example of the present subject matter.

FIG. 4(d) illustrates a ray diagram depicting an eye of the user and an arrangement of the obstacle, the objective lens, the relay lens, and the screen to display a refractive parameter value of −15 diopter, in accordance with an example of the present subject matter.

FIGS. 5(a)-5(c) illustrates an example display area of the screen of the handheld portable autorefractive device, in accordance with an example of the present subject matter.

FIGS. 6(a) and 6(b) illustrates a second example implementation of the handheld portable autorefractive device, in accordance with an example of the present subject matter.

FIG. 6(c) illustrates a pattern formed on a detector by a beam of light passing through a micro array, in accordance with an example implementation of the present subject matter.

FIG. 7 illustrates a first example method to detect refractive parameter of a user, in accordance with an example of the present subject matter.

FIG. 8 illustrates a second example method to detect refractive parameter of a user, in accordance with an example of the present subject matter.

### DETAILED DESCRIPTION

The present subject matter relates to a handheld portable autorefractive device for refraction techniques, such as an objective technique and a subjective technique and visual acuity detection. Conventionally, refraction techniques involve orthoptists, optometrists, and ophthalmologists to determine a subject's, alternatively referred to as a user's need for refractive correction by determining a spherical aberration component and/or a cylindrical aberration component of the refractive parameter of the eye. Optical instruments, such as phoropters, or Snellen charts are commonly used to detect the refractive parameter in subjective refraction techniques. Similarly, complex equipment such as a plurality of lenslet arrays, and the like are used in objective refraction techniques which are expensive. However, such techniques are restricted to be performed by a person skilled in the art.

In order to alleviate problems associated with the conventional techniques of refractive parameter detection and visual acuity detection, the present subject matter provides a handheld portable autorefractive device for refractive parameter detection and visual acuity detection, without professional intervention.

In operation, in one example implementation of the present subject matter, an initial pattern may be displayed on a screen of a handheld portable autorefractive device for detecting a refractive parameter of a user. A feedback from the user may be received through a feedback mechanism coupled to the screen, in response to the initial pattern displayed on the screen, where the user views the screen through a viewing unit. The viewing unit includes an obstacle, wherein the obstacle is to split the initial pattern displayed on the screen as visible to the user, to emulate a principle of diffraction of light. The screen is displaced from an initial position to a secondary position based on the feedback received from the user. The feedback from the user is received iteratively from the user and the screen is displaced to a final position until the initial pattern is correctly visible to the user. A refractive parameter is detected based on a displacement of the screen from the initial position to the final position.

In another example implementation of the present subject matter, a beam of light is directed towards an eye of a user. A reflected beam of light obtained from a retina of the eye of the user is deflected towards a micro array of a handheld portable autorefractive device. A pattern formed by the reflected beam of light passing through the micro array is detected to determine a distortion component of the pattern formed by the reflected beam of light, and a refractive parameter of the eye of the user is detected based on the distortion component.

The present subject matter thus provides an autorefractive device for refraction detection and vision acuity detection that is portable, cost-effective, and simple to use without professional intervention.

The above and other features, aspects, and advantages of the subject matter will be better explained with regard to the following description and accompanying figures. Wherever possible, the same reference numbers are used in the drawings and the following description to refer to the same or similar parts. While several examples are described, modifications, adaptations, and other implementations are possible.

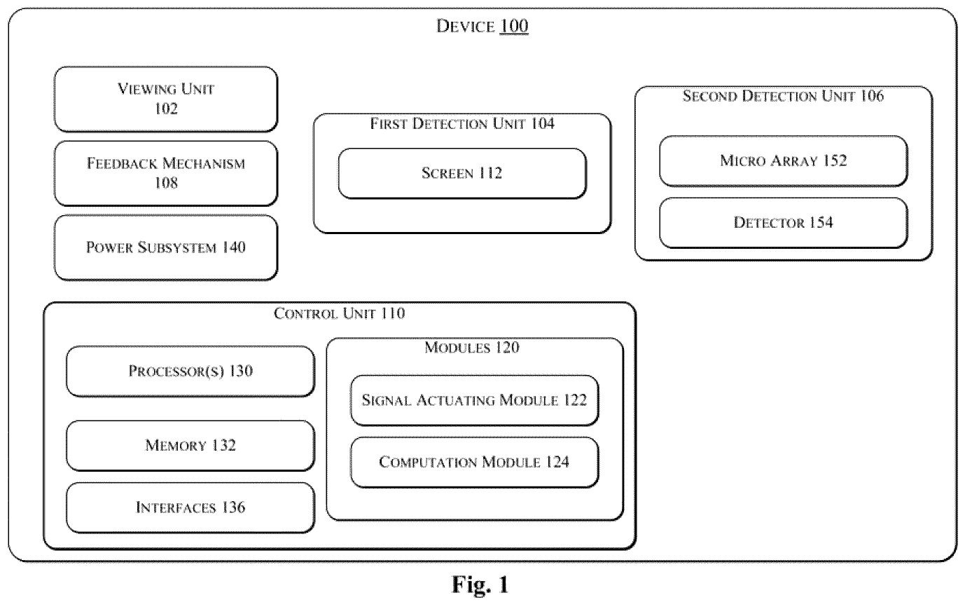

FIG. 1 illustrates a block diagram of a handheld portable autorefractive device 100, in accordance with an example of the present subject matter. The handheld portable autorefractive device 100, alternatively referred to as a device 100, may be configured to detect a refractive parameter and a visual acuity, such as a near vision acuity and a far vision acuity of an eye of a user, alternatively referred to as the user (not shown in the figure). The device 100 includes a viewing unit 102, a first detection unit 104, a second detection unit 106, a feedback mechanism 108, and a control unit 110. In one example, the user may view a screen 112 of the first detection unit 104 through the viewing unit 102. The control unit 110 of the device 100 may be configured to display a series of images or charts for detecting the refractive parameter and vision acuity of the eye of the user.

In an example, one or more modules 120 may be implemented to detect the refractive parameter, near vision acuity, and far vision acuity, of the user. The modules 120 may include a signal actuating module 122 and a computation module 124 which may be implemented as instructions executable by one or more processors. For instance, in the example where the control unit 110 of the device 100 performs a method for detecting the refractive parameter, the near vision acuity, and the far vision acuity of the user, the modules 120 are executed by a processor of the control unit 110. In case the method is implemented in part by the control unit 110 and in part by a server, the modules (depending on the step) will be distributed accordingly between the control unit 110 and the server.

In one example, the control unit 110 of the device 100 may be configured to receive input measurement signals from various measurement equipments of the device 100, such as the feedback mechanism 108, for example, and other measurement sensors. The control unit 110 may process the input signals obtained, with the help of a processor 130. The processor(s) 130 may be implemented as microprocessors, microcomputers, microcontrollers, digital signal processors, field programmable gate arrays (FPGA), central processing units, state machines, logic circuitries, and/or any devices that manipulate signals based on operational instructions. The functions of the various elements shown in the figure, including any functional blocks labelled as “processor(s)”, may be provided through the use of dedicated hardware as well as hardware capable of executing machine readable instructions. The processor(s) 130 may be implemented as a dedicated processor, a shared processor, or a plurality of individual processors, some of which may be shared, some of which may be on the device 100, and others may be on another device.

The control unit 110 may comprise a memory 132, that may be communicatively connected to the processor 130. Among other capabilities, the processor 130 may fetch and execute computer-readable instructions, stored in the memory 132. In one example, the memory 132 may store instructions that can be executed by the processor 130 to implement the signal actuating module 122 and the computation module 124. In other examples, instructions to implement the signal actuating module 122 and the computation module 124 may be stored in a memory outside of the device 100 in an external memory. The memory 132 may include any non-transitory computer-readable medium including, for example, volatile memory, such as RAM, or non-volatile memory, such as EPROM, flash memory, and the like. In an example, the method for detecting the refractive parameter of the user, near acuity, and far acuity of the user, may be performed by the control unit 110.

Further, the control unit 110 may comprise an interface(s) 136 to communicate the results obtained from the modules 120, for example, to a server. The interface(s) 136 may include a variety of computer-readable instructions-based interfaces and hardware interfaces that allow interaction with other communication, storage, and computing devices, such as network entities, web servers, databases, and external repositories, and peripheral devices. In one example, the refractive parameter values, and the like, may be viewed on a display screen (not shown in the figure) connected to the interface(s) 136 or integrated with the device 100. In one example, the refractive parameter value, the near acuity value, and the far acuity value computed may be shared to another device over a network (not shown in the figure). The network may be a wireless network or a combination of a wired and wireless network. The network can also include a collection of individual networks, interconnected with each other and functioning as a single large network, such as the Internet, Bluetooth, etc. Examples of such individual networks include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN), Long Term Evolution (LTE), and Integrated Services Digital Network (ISDN).

Further, the device 100 may include a power subsystem 140. The power subsystem 140 may include components to power the device 100 with a battery or a plurality of batteries. In another example, the power subsystem 140 may additionally or alternatively include components to power the device 100 using an AC voltage supply.

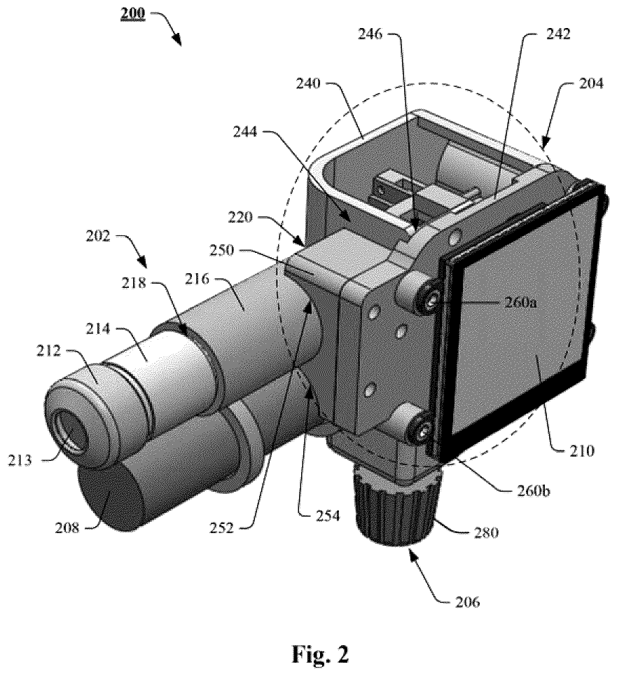

In a first example implementation of the device 100, the refractive parameter and visual acuity of the eye of the user may be detected based on a feedback provided by the user. In the first example implementation, an obstacle may be disposed in between an objective lens and a relay lens of the viewing unit 102. The objective lens, the obstacle, and the relay lens may be positioned coaxially in line with the screen 112 of the first detection unit 104, wherein the obstacle is to split an initial pattern displayed on the screen 112 as visible to the user, to emulate a principle of diffraction of light. In one example, the first detection unit 104 may be coupled to the feedback mechanism 108, through which the user may provide a feedback to the device 100. The first example implementation of the device 100 is discussed with reference to FIGS. 2-5.

In a second example implementation of the device 100, the refractive parameter and the visual acuity of the user may be detected automatically by the handheld portable autorefractive device 100. In one example, the device 100 in the second example implementation may include a second detection unit 106 coupled to the viewing unit 102. In one example, the second detection unit 106 may be positioned to receive a reflected beam of light from a beam splitter arrangement disposed in between the objective lens and the relay lens of the viewing unit 102. The reflected beam of light may be incident on a detector 154 after passing through a micro array 152 of the second detection unit 106 to form a pattern on the detector 154. In one example, the micro array 152 may include a plurality of micro openings, through which the reflected beam of light may pass through. Based on the pattern formed, the refractive parameter and vision acuity may be detected.

In a third example implementation of the device 100, the refractive parameter and visual acuity of the eye of the user may be detected in two modes of operation. In a first mode of operation, the device 100 may detect the refractive parameter and visual acuity of the eye of the user based on a feedback provided by the user. In a second mode of operation, the device 100 may be configured to detect the refractive parameter and visual acuity of the eye of the user automatically. In one example the first mode of operation and the second mode of operation may occur simultaneously. In another example, the first mode of operation and the second mode of operation may take place sequentially, in any order.

In one example, the refractive parameter values and visual acuity values collected from multiple users may be utilized for data mining, statistical analysis, for example, to provide data on prevalence and type of refractive parameters concerning to users from a geographical location, of various age groups, and the like.

FIG. 2 illustrates a perspective view of a first example implementation of the handheld portable autorefractive device 200, in accordance with an example of the present subject matter. The first example implementation of the handheld portable autorefractive device 200, alternatively referred to as a device 200, may be configured to detect a refractive parameter and a visual acuity, such as a near vision acuity and a far vision acuity of an eye of a user, alternatively referred to as the user. The device 200 includes a viewing unit 202, a first detection unit 204, a feedback mechanism 206, a control unit (not shown in the figure), and a power subsystem 208. In one example, the user may view a screen of the first detection unit 204 through the viewing unit 202. The control unit of the device 200 may be configured to display an image, pattern, chart, or characters, and the like, for detecting the refractive parameter and vision acuity of the eye of the user. In one example, the detection of vision acuity may include detection of near vision acuity and far vision acuity.

In an example, one or more modules (not shown in the figure) may be implemented to detect the refractive parameter, near vision acuity, and far vision acuity, of the user. The modules may include a signal actuating module and a computation module which may be implemented as instructions executable by one or more processors. For instance, in the example where the control unit of the device 200 performs a method for detecting the refractive parameter, the near vision acuity, and the far vision acuity of the user, the modules are executed by a processor of the control unit. In case the method is implemented in part by the control unit and in part by a server, the modules (depending on the step) will be distributed accordingly between the control unit and the server.

In one example, the control unit of the device 200 may be configured to receive input signals from various measurement equipments of the device 200, such as the feedback mechanism 206, for example, and other measurement sensors. The control unit may process the input signals obtained, with the help of a processor (not shown in the figure). The processor(s) may be implemented as microprocessors, microcomputers, microcontrollers, digital signal processors, field programmable gate arrays (FPGA), central processing units, state machines, logic circuitries, and/or any devices that manipulate signals based on operational instructions. The functions of the various elements shown in the figure, including any functional blocks labelled as “processor(s)”, may be provided through the use of dedicated hardware as well as hardware capable of executing machine readable instructions. The processor(s) may be implemented as a dedicated processor, a shared processor, or a plurality of individual processors, some of which may be shared, some of which may be on the device 200, and others may be on another device.

The control unit may comprise a memory (not shown in the figure), that may be communicatively connected to the processor. Among other capabilities, the processor may fetch and execute computer-readable instructions, stored in the memory. In one example, the memory may store instructions that can be executed by the processor to implement the signal actuating module. In other examples, instructions to implement the computation module may be stored in a memory outside of the device 200 in an external memory. The memory may include any non-transitory computer-readable medium including, for example, volatile memory, such as RAM, or non-volatile memory, such as EPROM, flash memory, and the like. In an example, the method for detecting the refractive parameter of the user, near acuity, and far acuity of the user, may be performed by the control unit.

Further, the control unit may comprise an interface(s) (not shown in the figure) to communicate the results obtained from the modules, for example, to a server. The interface(s) may include a variety of computer-readable instructions-based interfaces and hardware interfaces that allow interaction with other communication, storage, and computing devices, such as network entities, web servers, databases, and external repositories, and peripheral devices. In one example, the refractive parameter values, and the like, may be viewed on a display screen 210 connected to the interface(s) or integrated with the device 200. In one example, the refractive parameter value, the near acuity value, and the far acuity value computed may be shared to another device over a network (not shown in the figure). The network may be a wireless network or a combination of a wired and wireless network. The network can also include a collection of individual networks, interconnected with each other and functioning as a single large network, such as the Internet, Bluetooth, etc. Examples of such individual networks include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN), Long Term Evolution (LTE), and Integrated Services Digital Network (ISDN).

In one example, the viewing unit 202 and the power subsystem 208 may be positioned parallelly adjacent to one another, such that the viewing unit 202 may be positioned along a first longitudinal axis and the power subsystem 208 may be positioned along a second longitudinal axis. In one example, the first longitudinal axis may lie above the second longitudinal axis.

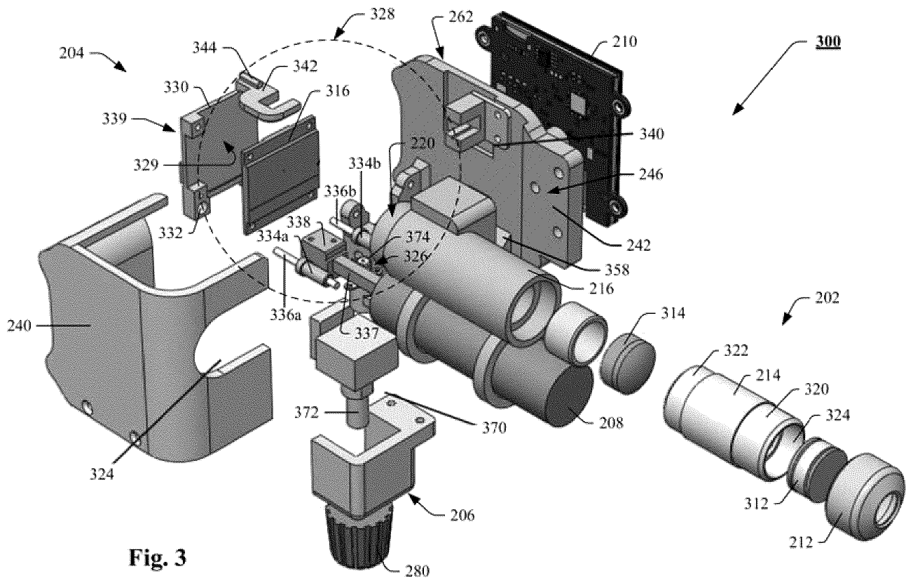

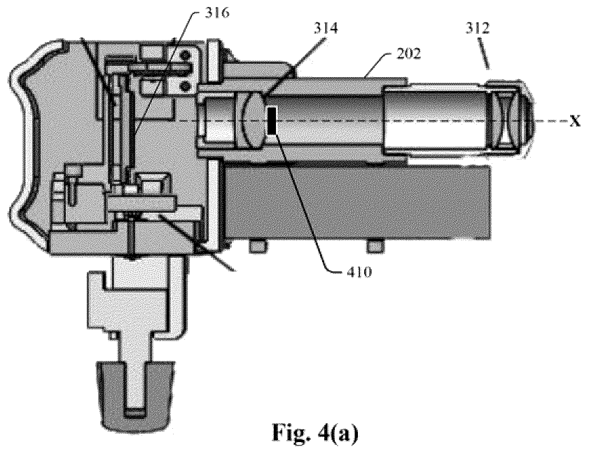

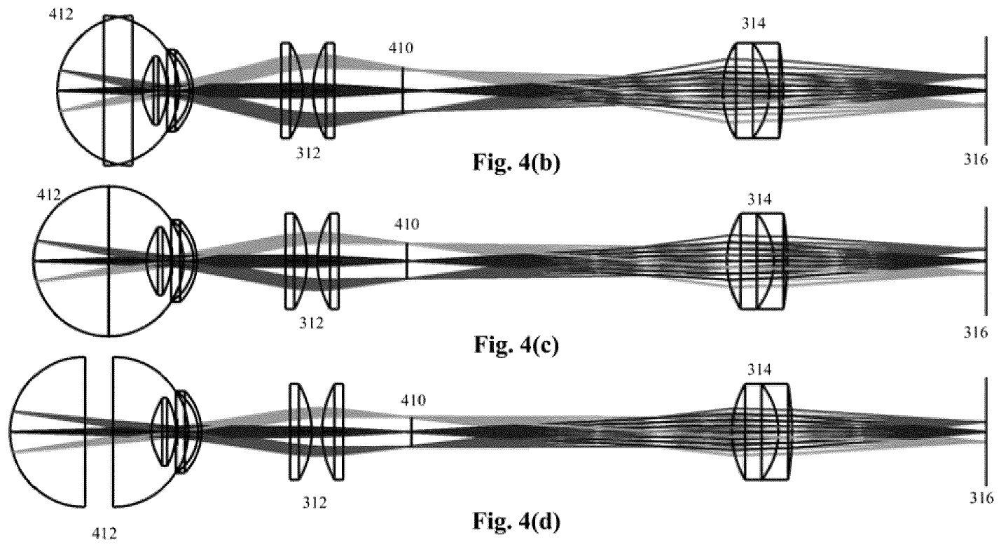

Further, the viewing unit 202 includes an eye piece 212, a spacer 214, and a housing element 216. The eye piece 212 may be provided with an aperture 213 through which the user may view the screen of the first detection unit 204. The eye piece 212 may be coupled to the spacer 214. In one example, the spacer 214 may be a hollow structure, where an objective lens (not shown in the figure) may be disposed. One end of the spacer 214 may be connected to the eye piece 212 and the other end of the spacer 214 may be connected to a first end 218 of the housing element 216. Similar to the spacer 214, the housing element 216 may also be a hollow structure to accommodate a relay lens and an obstacle (not shown in the figure). In one example, the housing element 216 may be cylindrical in structure. Further, a second end 220 of the housing element 216 may be partially disposed into the first detection unit 204. In one example, the eye piece 212, the spacer 214, and the housing element 216 of the viewing unit 202 may be coaxially aligned along a longitudinal axis of the screen of the first detection unit 204. The construction of the viewing unit 202 is discussed in detail with reference to FIGS. 3 and 4.

In one example, the first detection unit 204 includes a first plate 240 and a second plate 242. The first plate 240 may be a U-shaped plate provided with a slot (not shown in the figure) at a front end 244. The slot is to receive the second end 220 of the housing element 216 of the viewing unit 202. In one example, the first plate 240 and the second plate 242 may be so arranged, in order to form an enclosure. The enclosure formed, may house various components of the first detection unit 204, such as the screen, a motor, and an actuating mechanism. In one example, a first edge and a second edge (not shown in the figure) of the first plate 240 may be attached to a first side 246 of the second plate 242 to form the enclosure. In one example, the second plate 242 may protrude beyond a portion where the first edge of the first plate 240 and the second plate 242 are connected, to accommodate a bracket 250.

The bracket 250 may be mounted on the second plate 242 in a direction substantially perpendicular to the second plate 242, where the bracket 250 may include a first arcuate surface 252 and a second arcuate surface 254. The first arcuate surface 252 may be provided to support the housing element 216 of the viewing unit 202. Similarly, the second arcuate surface 254 may be provided to support to the power subsystem 208. In one example, the shape and surface area of the first arcuate surface 252 and the second arcuate surface 254 may be designed based on the shape and size of the viewing unit 202 and the power subsystem 208, respectively. In one example, mechanical fasteners 260a and 260b, such as screws, bolts, studs, or the like may be used to mount the bracket 250 on to the second plate 242. In one example, a second side of the second plate 242 may be provided with the display screen 210. The display screen 210 may be configured to display the refractive parameter values and the vision acuity values detected.

In one example, the screen of the first detection unit 204 may be coupled to the feedback mechanism 206. The user may provide an input for detecting the refractive parameter through the feedback mechanism 206. In one example, but not limited to, the feedback mechanism 206 may include a focus knob 280, through which an input to the device 200 may be provided. For example, the user may rotate the focus knob 280 in response to an initial pattern displayed on the screen. In response to the initial pattern displayed on the screen, the user may provide a feedback, where the user views the screen through the viewing unit 202 which includes an obstacle. The obstacle is to split the initial pattern displayed on the screen as visible to the user, to emulate a principle of diffraction of light. In one example, the screen may be displaced from and initial position to a secondary position based on the feedback received from the user. The user may iteratively provide feedback to the device 200 to displace the screen to a final position until the initial pattern is correctly visible to the user. In one example, the refractive parameter may be detected based on a displacement of the screen from the initial position to the final position.

Further, in one example, on detecting the refractive parameter, steps to detect vision acuity may be performed, where the vision acuity detection may include detection of near vision acuity and far vision acuity, collectively and alternatively referred to as vision acuity. In one example, in order to detect the near vision acuity, the control unit may be configured to displace the screen to a first position, where the first position is at a first pre-determined distance from the objective lens of the hand-held portable autorefractive device 200. In one example, the first-predetermined distance may be equivalent to a range between 3 m to 4 m from the objective lens. On positioning the screen at the first position, the control unit may be configured to display a near vision acuity chart on the screen. On viewing the near vision acuity chart the user may provide a second feedback to the device 200. In one example, the third feedback may be provided through the feedback mechanism 206. In one example, the second feedback may be associated with the user identifying characters from the near vision acuity chart clearly. In one example, the user may provide the second feedback until the user is able to identify characters, for example, on the near vision acuity chart. Although the following description uses an example of the near vision acuity chart including characters, any image, pattern, and the like may be displayed. In one example, the screen may be displaced from the first position to a tertiary position, based on the second feedback provided by the user and a near vision acuity of an eye of the user may be computed based on a distance of the screen displaced from the first position to the tertiary position.

In one example, in order to detect the far vision acuity, the control unit may be configured to displace the screen to a second position, where the second position is at a second pre-determined distance from the objective lens of the hand-held portable autorefractive device 200. In one example, the second-predetermined distance may be equivalent to a range between 3 m to 4 m from the objective lens. On positioning the screen at the second position, the control unit may be configured to display a far vision acuity chart on the screen. On viewing the far vision acuity chart the user may provide a third feedback to the device 200, where the third feedback may be associated with the user identifying characters from the far vision acuity chart clearly. In one example, the third feedback may be provided through the feedback mechanism 206. In one example, the user may provide the third feedback until the user is able to identify characters, for example, on the far vision acuity chart. Although the following description uses an example of the far vision acuity chart including characters, any image, pattern, and the like may be displayed. In one example, the screen may be displaced from the second position to a fourth position, based on the third feedback provided by the user and a far vision acuity of an eye of the user may be computed based on a distance of the screen displaced from the second position to the fourth position. The construction and working of the device 200 are discussed in detail with reference to FIG. 3.

FIG. 3 illustrates an exploded view 300 of the handheld portable autorefractive device 200, in accordance with an example of the present subject matter. As depicted in FIG. 3, the viewing unit 202 includes the eye piece 212, the objective lens 312, the spacer 214, the housing element 216, and a relay lens 314. An obstacle (not shown in the figure) may be disposed in between the objective lens 312 and the relay lens 314. In one example, the eye piece 212, the objective lens 312, the spacer 214, the relay lens 314, and the housing element 216 may be coaxially aligned along a longitudinal axis of a screen 316, such that when the user (not shown in the figure) looks through the eye piece 212 of the viewing unit 202, a display area (not shown in the figure) of the screen 316 may be visible.

In one example, the spacer 214 may be disposed in between the eye piece 212 and the housing element 216. In one example, the objective lens 312 may be positioned in a first portion 320 of the spacer 214, such that an inner surface of the spacer 214 is in contact with an outer surface of the objective lens 312. Further, a second portion 322 of the spacer 214 may be connected to the housing element 216. In one example, the relay lens 314 may be disposed in the housing element 216 of the viewing unit 202, and the obstacle may be disposed in between the objective lens 312 and the relay lens 314 as depicted in FIG. 4. On continuing with the description of FIG. 3, in one example, the second end 220 of the housing element 216 may be partially disposed into a slot 324 of the first plate 240 of the first detection unit 204 to couple the viewing unit 202 to the first detection unit 204.

The first detection unit 204 includes the screen 316, a motor 326, and an actuating mechanism 328. In one example, the screen 316 may be an LED display or an OLED display, on which images or charts may be displayed to detect the refractive parameter and visual acuity. In one example, the screen 316 may be mounted on a first surface 329 of a support plate 330. The support plate 330 may be provided with one or more slots 332 to allow one or more guiding sleeves 334a and 334b of a guiding element of the actuating mechanism 328 to pass through. The one or more guiding sleeves 334a and 334b may be provided to facilitate a controlled movement of the support plate 330. In one example, the one or more guiding sleeves 334a and 334b may include an opening to allow the guiding element 336a and 336b to pass through, respectively.

The first guiding element 336a and the second guiding element 336b, collectively referred to as guiding elements 336, may be positioned substantially parallel to one another. In one example, a distance between the first guiding element 336a and the second guiding element 336b may be equal to a width of the support plate 330. The support plate 330 may be mounted substantially perpendicular to the one or more guiding elements 336.

In one example, the support plate 330 may be mounted at a distal end of a ridged bar 337 of the actuating mechanism 328, such that a displacement of the ridged bar 337 causes the support plate 330 and the screen 316 to move along a longitudinal axis of the screen 316 in the forward and backward direction to detect the refractive parameter and visual acuity of the user. In one example, the ridged bar 337 includes a head block 338 that may be in contact with a second surface 339 of the support plate 330, in order to support the support plate 330. In one example, the movement of the support plate 330 along the guiding elements 336 may be limited by a limit switch 340.

In one example, the limit switch 340 may be provided on the first side 246 of the second plate 242 of the first detection unit 204. The limit switch 340 may be provided for device calibration, where the limit switch 340 may be provided to restrict the movement of the screen 316 along the longitudinal axis of the screen 316. In one example, the movement of the screen 316 along the longitudinal axis of the screen 316 can be varied to detect the refractive parameter in a range of +15 to −15 diopters.

Further, a limit hook 342 may be provided on the support plate 330. The limit hook 342 may be coupled to the support plate 330 with a sliding element 344. The sliding element 344 is to slide along a first groove (not shown in the figure) provided on the first side 246 of the second plate 242 of the first detection unit 204, causing a movement of the limit hook 342. The movement of the limit hook 342 may be restricted by the limit switch 340. The restriction in the movement of the limit hook 342, in turn restricts the movement of the support plate 330.

Further, the movement of the screen 316 coupled to the support plate 330 may be actuated by the motor 326. In one example, the motor 326 may be a stepper motor. The motor 326 may receive actuating signals from the signal actuating module of the control unit. The actuating signals from the signal actuating module may be based on an input provided by the user through the focus knob 280 of the feedback mechanism 206. In one example, the feedback mechanism 206 of the device 200 may further include an encoder 370 coupled to the focus knob 280.

In one example, the encoder 370 may be an optical encoder 370. An optical encoder is a sensing device in which a mechanical movement of a shaft of the encoder can be tracked and converted into an encoding signal. In one example, the focus knob 280 may be coupled to a shaft 372 of the optical encoder 370. Based on the rotation of the focus knob 280, the shaft 372 of the optical encoder 370 rotates, to generate an encoding signal. In one example, the encoding signal may correspond to a refractive parameter value.

Further, the control unit may obtain the encoding signal and generate an actuating signal to drive the motor 326. The actuating signal generated by the control unit is to cause the motor 326 to rotate. When the motor 326 rotates, a top gear 374 of the motor 326 rotates. The top gear 374 may be operatively coupled to the ridged bar 337 of the actuating mechanism 328. The teeth of the top gear 374 may engage with a plurality of ridges provided on the ridged bar 337, to transfer torque from the motor 326 to the ridged bar 337 causing the ridged bar 337 to be displaced along a longitudinal axis, in turn displacing the screen 316 along the longitudinal axis of the screen 316. Based on a distance of the displacement of the screen 316 from one position to another, the refractive parameter and vision acuity may be detected. Thus, the present subject matter facilitates in accurate detection of a refractive parameter value, particularly based on the feedback provided by the subject.

FIG. 4(a) illustrates a sectional view of the first example implementation of the handheld portable autorefractive device 200, in accordance with an example of the present subject matter. As depicted in FIG. 4(a), in addition to the objective lens 312 and the relay lens 314, an obstacle 410 may be disposed in the viewing unit 202, in between the objective lens 312 and the relay lens 314. In one example, the obstacle 410 may be positioned at a fixed distance from the relay lens 314 along a primary axis X. The obstacle 410 may split an initial pattern displayed on the screen 316 as perceived by the user, to emulate a principle of diffraction of light. In one example, the screen 316 may move a total of 3 mm to display a refractive parameter value ranging between +15 diopters to −15 diopters. FIGS. 4(b) to 4(d) illustrate ray diagrams depicting an arrangement of the obstacle, the objective lens, the relay lens, and the screen to display a refractive parameter values ranging between +15 diopters to −15 diopters based on a position of screen 316.

FIG. 4(b) illustrates a ray diagram depicting an eye 412 of the user and an arrangement of the obstacle, the objective lens, the relay lens, and the screen to display a refractive parameter value of +15 diopter, in accordance with an example of the present subject matter.

FIG. 4(c) illustrates a ray diagram depicting an eye 412 of the user and an arrangement of the obstacle, the objective lens, the relay lens, and the screen to display a refractive parameter value of 0 diopter, in accordance with an example of the present subject matter.

FIG. 4(d) illustrates a ray diagram depicting an eye 412 of the user and an arrangement of the obstacle, the objective lens, the relay lens, and the screen to display a refractive parameter value of −15 diopter, in accordance with an example of the present subject matter.

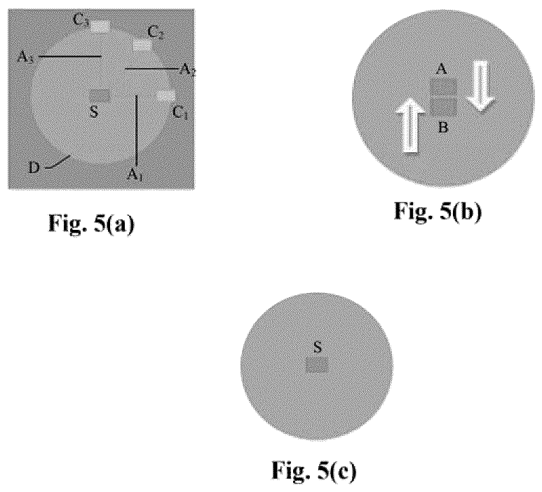

FIG. 5 illustrates an example display area of the screen of the handheld portable autorefractive device, in accordance with an example of the present subject matter. While an implementation of the method for detecting the refractive parameter has been explained with an example implementation as described below, it is to be understood that other variations to detect the refractive parameter is possible. The description is not to be construed as limited to the example implementation described. In one example, to detect the refractive parameter of an eye of a user, a plurality of patterns, images, characters, and the like may be displayed on the screen 316.

To detect the refractive parameter of the user, the user may look through the eye piece provided in the viewing unit of the device. When the user looks into the eye piece of the viewing unit, the user may be able to view a display area D of the screen. In one example, the default position of the screen may be set to a centre of the guiding element of the actuating mechanism. The refractive parameter is detected based on the principle of diffraction. When the handheld portable autorefractive device is switched on, the control unit may be configured to display an initial pattern on the screen. In one example, the initial pattern may be displayed at a centre of the display area D.

Based on the initial pattern displayed on the screen, the user may provide a feedback in response to the initial pattern displayed on the screen through the focus knob. In one example, the initial pattern S may be displayed on the screen at a first position on the display area D. When the user views the initial pattern displayed, the user may either be able to view the initial pattern exactly as displayed, or the user may be able to a split in the initial pattern. For example, the user may view S as one unit as shown in FIG. 5(a) or may be able to view two units A and B as shown in FIG. 5(b). The split in the initial pattern S causing the user to see two units A and B is due to the obstacle that is positioned in between the objective lens and the relay lens, causing diffraction of light. Based on whether the user is able to view the initial pattern S without the split or with the split, the user may provide a feedback through the focus knob, or a switch, and the like. Based on the feedback provided by the user in response to the initial pattern S displayed on the screen, the refractive parameter may be detected.

On detection of the refractive parameter, based on a position of the initial pattern displayed on the display area D, a spherical aberration component, a cylindrical aberration component, and an axial aberration component may be computed. For example, if the initial pattern S is displayed at the centre of the display area, the spherical aberration component of the refractive parameter may be computed and if the initial pattern is displayed along a circumference of the display area D, at an axis, for example, the cylindrical component and an axial component of the refractive parameter may be computed.

In a scenario, where the user does not see a split in the initial pattern S and views the initial pattern as is, the user may provide a feedback through the switch, based on which it may be understood that the refractive parameter at that point is zero. For example, if the initial pattern is displayed at the centre of the display area D and the user does not see a split in the initial pattern S, it may be understood that the spherical aberration component of the refractive parameter is zero. However, in a scenario where the user sees the initial pattern S as two units A and B as shown in FIG. 5(b), the user may adjust the focus knob by rotating it to cause the screen to be displaced. In one example, the screen may be displaced along a longitudinal axis of the screen, such that the screen moves in a forward direction, or a backward direction based on the rotation of the focus knob. Rotation of the focus knob causes the two units A and B to move with respect to one another as perceived by the user. The user may iteratively provide feedback to displace the screen to a final position until the initial pattern is correctly visible to the user as S′. The encoder coupled to the focus knob may generate the encoding signal to displace the screen from the initial position to the final position. At the final position, the initial pattern may be a result of the merging of the two units A and B as shown in FIG. 5(c). Once the user is able to view the initial pattern correctly, the user may provide a feedback through the switch. Further, based on the detection of refractive parameter, the spherical aberration component of the refractive parameter may be computed, which may be displayed on the display screen. Similarly, the initial pattern may be displayed at positions T₁, T₂, and T₃, along a first axis A₁, a second axis A₂, and a third Axis A₃, respectively, where the steps discussed above may be repeated to compute the cylindrical aberration component and axial component of the refractive parameter.

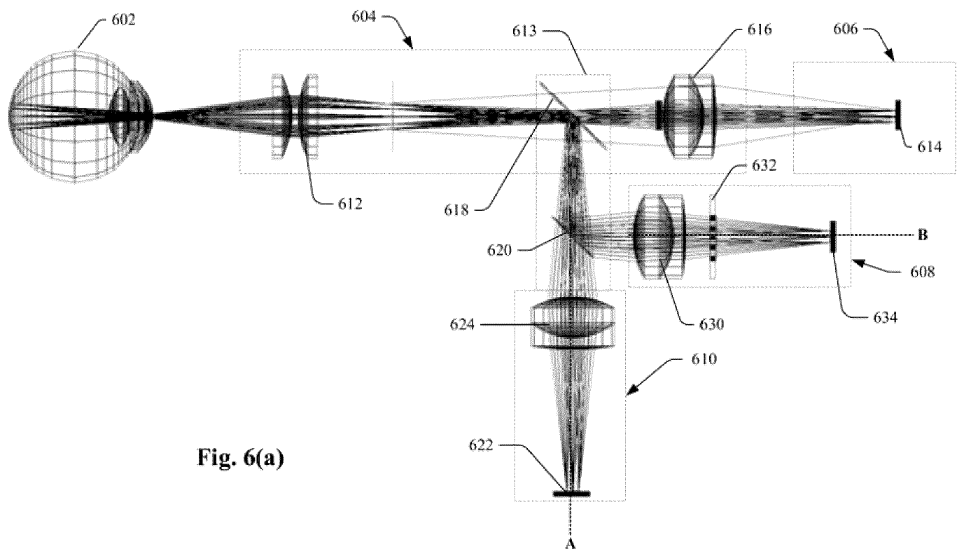

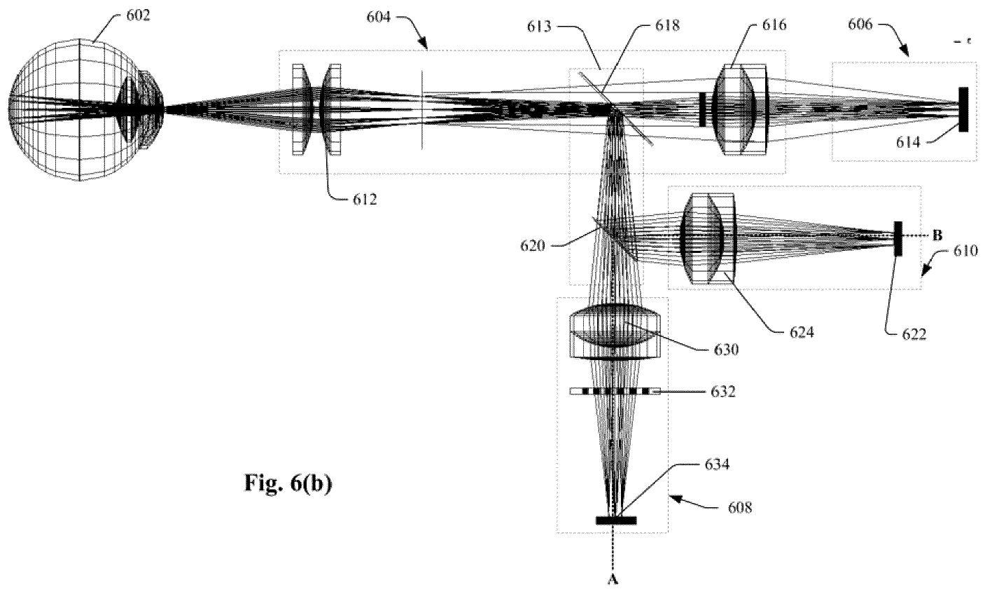

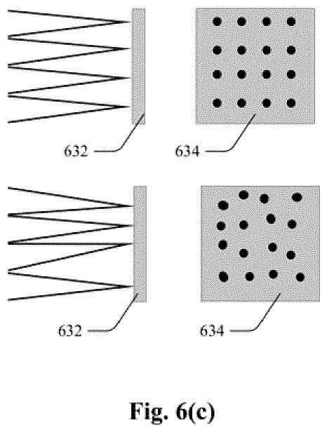

FIG. 6(a) illustrates a second example implementation of the handheld portable autorefractive device 600, in accordance with an example of the present subject matter. The handheld portable autorefractive device 600, alternatively referred to as a device 600, may be configured to detect a refractive parameter and a visual acuity, such as a near vision acuity and a far vision acuity of an eye of a user, alternatively referred to as the user 602. The device 600 includes a viewing unit 604, a first detection unit 606, a second detection unit 608, a light source unit 610, and a control unit (not shown in the figure). In one example, the user 602 may view a screen 614 of the first detection unit 606 through the viewing unit 604. The control unit of the device 600 may be configured to display an image, pattern, chart, or characters, and the like for detecting the refractive parameter and vision acuity of the eye of the user. In one example, the detection of vision acuity may include detection of near vision acuity and far vision acuity.

In an example, one or more modules (not shown in the figure) may be implemented to detect the refractive parameter, near vision acuity, and far vision acuity, of the user. The modules may include a computation module which may be implemented as instructions executable by one or more processors. For instance, in the example where the control unit of the device 600 performs a method for detecting the refractive parameter, the near vision acuity, and the far vision acuity of the user, the modules are executed by a processor of the control unit. In case the method is implemented in part by the control unit and in part by a server, the modules (depending on the step) will be distributed accordingly between the control unit and the server.

In one example, the control unit of the device 600 may be configured to receive input signals from various measurement equipments of the device 600, such as the second detection unit 608, for example, and other measurement sensors. The control unit may process the input signals obtained, with the help of a processor (not shown in the figure). The processor(s) may be implemented as microprocessors, microcomputers, microcontrollers, digital signal processors, field programmable gate arrays (FPGA), central processing units, state machines, logic circuitries, and/or any devices that manipulate signals based on operational instructions. The functions of the various elements shown in the figure, including any functional blocks labelled as “processor(s)”, may be provided through the use of dedicated hardware as well as hardware capable of executing machine readable instructions. The processor(s) may be implemented as a dedicated processor, a shared processor, or a plurality of individual processors, some of which may be shared, some of which may be on the device 600, and others may be on another device.

The control unit may comprise a memory (not shown in the figure), that may be communicatively connected to the processor. Among other capabilities, the processor may fetch and execute computer-readable instructions, stored in the memory. In one example, the memory may store instructions that can be executed by the processor to implement the computation module. In other examples, instructions to implement the computation module may be stored in a memory outside of the device 600 in an external memory. The memory may include any non-transitory computer-readable medium including, for example, volatile memory, such as RAM, or non-volatile memory, such as EPROM, flash memory, and the like. In an example, the method for detecting the refractive parameter of the user, near acuity, and far acuity of the user, may be performed by the control unit.

Further, the control unit may comprise an interface(s) (not shown in the figure) to communicate the results obtained from the modules, for example, to a server. The interface(s) may include a variety of computer-readable instructions-based interfaces and hardware interfaces that allow interaction with other communication, storage, and computing devices, such as network entities, web servers, databases, and external repositories, and peripheral devices. In one example, the refractive parameter values, and the like, may be viewed on a display screen (not shown in the figure) connected to the interface(s) or integrated with the device 600. In one example, the refractive parameter value, the near acuity value, and the far acuity value computed may be shared to another device over a network (not shown in the figure). The network may be a wireless network or a combination of a wired and wireless network. The network can also include a collection of individual networks, interconnected with each other and functioning as a single large network, such as the Internet, Bluetooth, etc. Examples of such individual networks include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN), Long Term Evolution (LTE), and Integrated Services Digital Network (ISDN).

Further, the device 600 may include a power subsystem (not shown in the figure). The power subsystem may include components to power the device 600 with a battery or a plurality of batteries. In another example, the power subsystem may additionally or alternatively include components to power the device using an AC voltage supply.

In one example, the first detection unit 606 and the second detection unit 608 may be similar to the first detection unit 104 and the second detection unit 106 as explained with reference to FIG. 1.

In one example, a user may look at the screen 614 of the first detection unit 606 through the viewing unit 604 of the device 600, where an initial pattern may be displayed. In one example, the viewing unit 604 may include an objective lens 612, a beam splitter arrangement 613 and a relay lens 616 arranged along an axis of the screen 614, where the objective lens 612, the beam splitter arrangement 613, and the relay lens 616 are coaxial to one another. In one example, the beam splitter arrangement 613 may be disposed in between the objective lens 612 and the relay lens 616. Further, the beam splitter arrangement 613 may include a first beam splitter 618 and a second beam splitter 620, where the first beam splitter 618 and the second beam splitter 620 may be positioned along a vertical axis separated from one another at a predefined distance. In one example, the first beam splitter 618 may be disposed in between the objective lens 612 and the relay lens 616 of the viewing unit 604 and the second beam splitter 620 may be substantially perpendicular to the first beam splitter 618.

In one example, the second beam splitter 620 may be coupled to a light source unit 610 along a first axis A and the second detection unit 608 along a second axis B, where the first axis A and the second axis B may be substantially perpendicular to one another. In one example, the position of the light source unit 610 and the second detection unit 608 may be interchanged as depicted in FIG. 6(b). In one example, the light source unit 610 may include a light source 622 and a light source lens 624. The light source lens 624 may be positioned in between the second beam splitter 620 and the light source 622. In one example, but not limited to, the light source 622 may be an LED.

Further, in one example, the second detection unit 608 includes a detector lens 630 disposed in between the beam splitter arrangement 613 and a micro array 632, where the detector lens 630 may be a singlet lens, a doublet lens, or a combination of a singlet lens and a doublet lens. In one example, the detector lens 630 may be positioned adjacent to the second beam splitter 620 of the beam splitter arrangement 613. The second detection unit 608 may further include a detector 634, such that the micro array 632 may be positioned in between the detector lens 630 and the detector 634. In one example, but not limited to, the micro array may include a plurality of micro-openings, where a shape of a plurality of micro-openings of the micro array 632 may be any one of a circular shape, an oval shape, a square shape, a rectangular shape, and the like. In one example, the second beam splitter 620, the detector lens 630, the micro array 632, and the detector 634 may be coaxial to one another.

In one example, an initial pattern may be displayed on the screen 614 of the first detection unit 606. Once the user views the initial pattern displayed on the screen 614 through the viewing unit 604, in one example, as shown in the figure, a beam of light may be directed towards the eye of the user 602. In one example, the beam of light may be emitted from the light source 622 of the light source unit 610. The beam of light emitted, may travel through the light source lens 624 to be incident on the second beam splitter 620. The light rays incident on the second beam splitter 620 may then be reflected and incident on the first beam splitter 618, from where the light rays may be reflected through the objective lens 612 of the viewing unit 604 to be incident on a retina of the eye of the user 602.

Further, a reflected beam of light received from the retina of the eye of the user 602 may be deflected towards the micro array 632 of the device 600. Light rays received from the retina of the user 602 may thus be passed through the detector lens 630 and the micro array 632 to be incident on the detector 634. In one example, the micro array 632 may include micro-openings for the reflected beam of light to pass through. In one example, the reflected beam of light passing through the micro array 632 may form a pattern on the detector 634 as shown in FIG. 6(c). FIG. 6(c) illustrates, and example pattern formed on the detector 634, when a reflected beam of light passes through the micro array 632, in accordance with an example of the present subject matter. In FIG. 6(c) a micro-opening of the micro array 632 is in the shape of a micro pin hole. However, other shapes of micro-openings are possible. In one example, a distortion component of the pattern formed on the detector 634 may be calculated by methods known in the art, such as based on Hartmann Shak principle. Based on the distortion component computed, the refractive parameter of the eye of may be detected.

In one example, on detecting the refractive parameter, a vision acuity may be detected. Detection of vision acuity may include a near vision acuity detection and a far vision acuity detection. In one example, the screen 614 of the device 600 may be positioned at a first position (not shown in the figure), where the first position is at a first pre-determined distance from the objective lens 612 of the device 600. In one example, a near vision acuity chart may be displayed on the screen 614. In one example, a near vision acuity chart may include characters for identification. Although the following description uses an example of the near vision acuity chart including characters, any image, pattern, and the like may be displayed.

On displaying the near vision acuity chart on the screen 614, a beam of light from the light source unit 610 may be directed towards an eye of the user 602, as explained above. The reflected beam of light received from the retina of the eye of the user 602 may be directed towards the micro array 632 to be incident on the detector 634, where the reflected beam of light forms a pattern on passing through the micro array 632. On detecting the pattern formed on the detector 634, a distortion component of the pattern formed may be calculated based on which, the near vision acuity may be detected.

Similarly, a far vision acuity of the user may be detected, where the screen 614 of the device 600 may be positioned at a second position (not shown in the figure), where the second position is at a second pre-determined distance from the objective lens 612 of the device 600. In one example, a far vision acuity chart may be displayed on the screen 614. In one example, the far vision acuity chart may include characters for identification. Although the following description uses an example of the far vision acuity chart including characters, any image, pattern, and the like may be displayed. On displaying the far vision acuity chart on the screen, a light beam may be directed towards an eye of the user as explained above. The reflected beam of light received from the retina of the eye of the user 602 may be directed towards the micro array 632 to be incident on the detector 634, where the reflected beam of light forms a pattern on passing through the micro array 632. On detecting the pattern formed on the detector 634, a distortion component of the pattern formed may be calculated based on which, the far vision acuity may be detected.

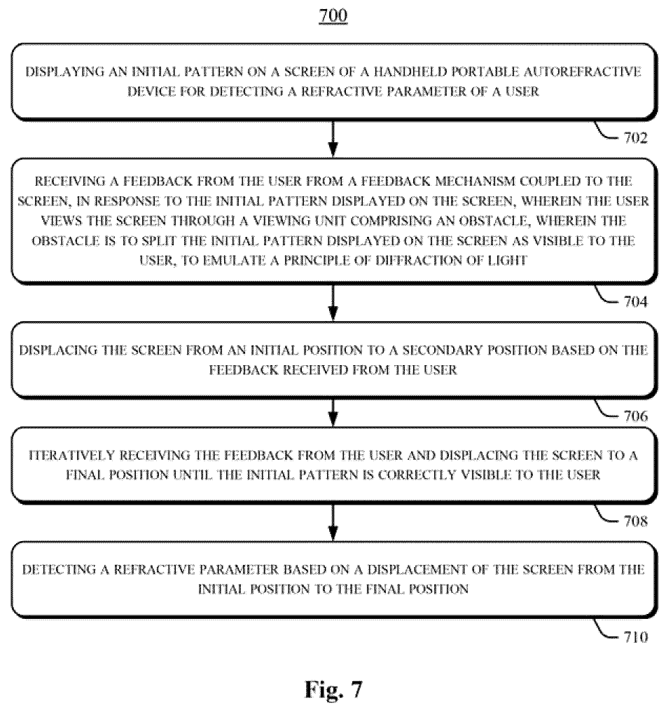

FIG. 7 illustrates a first example method to detect refractive parameter of a user, in accordance with an example of the present subject matter. The order in which the method 700 is described is not intended to be construed as a limitation, and any number of the described method blocks can be combined in any order to implement method 700 or an alternative method. Additionally, individual blocks may be deleted from the method 700 without departing from the spirit and scope of the subject matter described herein. Furthermore, the method 700 may be implemented in any suitable hardware, computer readable instructions, firmware, or combination thereof. For discussion, the method 700 is described with reference to the implementations illustrated in FIGS. 2-5(a)-5(c).

At block 702, an initial pattern is displayed on a screen of a handheld portable autorefractive device to detect a refractive parameter of a user.

At block 704, a feedback from the user in response to the initial pattern displayed on the screen may be received through a feedback mechanism coupled to the screen. Where, the user views the screen through a viewing unit which includes an obstacle, where the obstacle is to split the initial pattern displayed on the screen as visible to the user, to emulate a principle of diffraction of light.

At block 706, the screen is displaced from an initial position to a secondary position based on the feedback received from the user.

At block 708, the feedback from the user is iteratively received to displace the screen to a final position until the initial pattern is correctly visible to the user.

At block 710, a refractive parameter is detected based on a displacement of the screen from the initial position to the final position.

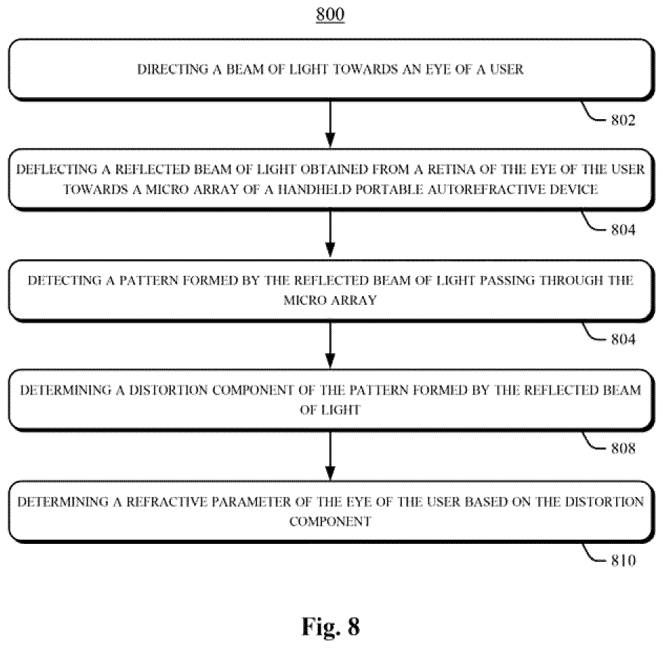

FIG. 8 illustrates a second example method to detect refractive parameter of a user, in accordance with an implementation of the present subject matter. The order in which the method 800 is described is not intended to be construed as a limitation, and any number of the described method blocks can be combined in any order to implement method 800 or an alternative method. Additionally, individual blocks may be deleted from the method 800 without departing from the spirit and scope of the subject matter described herein. Furthermore, the method 800 may be implemented in any suitable hardware, computer readable instructions, firmware, or combination thereof. For discussion, the method 800 is described with reference to the implementations illustrated in FIGS. 6(a)-6(c).

At step 802, a beam of light is directed towards an eye of the user.

At step 804, a reflected beam of light obtained from a retina of the eye of the user is deflected towards a micro array of a hand-held portable autorefractive device.

At block 806, a pattern formed by the reflected light beam passing through the micro array is detected. In one example, a shape of a plurality of micro openings of the micro array is any one of a circular shape, an oval shape, a square shape, or a rectangular shape.

At block 808, a distortion component of the pattern formed by the reflected light beam is determined.

At block 810, a refractive parameter of the eye of the user is detected based on the distortion component.

Although the present subject matter has been described with reference to specific implementations, this description is not meant to be construed in a limiting sense. Various modifications of the disclosed implementations, as well as alternate implementations of the subject matter, will become apparent to persons skilled in the art upon reference to the description of the subject matter.

1. A method comprising:
displaying an initial pattern on a screen of a handheld portable autorefractive device for detecting a refractive parameter of a user;
receiving a feedback from the user from an input coupled to the screen, in response to the initial pattern displayed on the screen, wherein the user views the screen through a viewer comprising an obstacle, wherein the obstacle is to split the initial pattern displayed on the screen as visible to the user, to emulate a principle of diffraction of light;
displacing the screen from an initial position to a secondary position based on the feedback received from the user;
iteratively receiving the feedback from the user and displacing the screen to a final position until the initial pattern is correctly visible to the user;
and detecting a refractive parameter based on a displacement of the screen from the initial position to the final position.

2. (canceled)

3. The method as claimed in claim 1, further comprising:
positioning the screen at a first position, wherein the first position is at a first pre-determined distance from an objective lens of the handheld portable autorefractive device;
displaying a near vision acuity chart on the screen;
obtaining a second feedback from the user, wherein the second feedback is associated with the user identifying characters from the near vision acuity chart;
displacing the screen from the first position to a tertiary position, based on the second feedback provided by the user;
and computing a near vision acuity of an eye of the user based on a distance of the screen displaced from the first position to the tertiary position.

4. The method as claimed in claim 3, wherein the first pre-determined distance is equivalent to a range between 3 m to 4 m from the objective lens.

5. The method as claimed in claim 1, further comprising:
positioning the screen at a second position, wherein the second position is at a second pre-determined distance from an objective lens of the handheld portable autorefractive device;
displaying a far vision acuity chart on the screen;
obtaining a third feedback from the user, wherein the third feedback is associated with the user identifying characters from the far vision acuity chart;
displacing the screen from the second position to a fourth position, based on the third feedback provided by the user;
and computing a far vision acuity of an eye of the user based on a distance of the screen displaced from the second position to the fourth position.

6. The method as claimed in claim 5, wherein the second pre-determined distance is equivalent to a range between 3 m to 4 m from the objective lens.

7. A handheld portable autorefractive device comprising:
a screen;
a controller configured to display an initial pattern on the screen of the screen for a user to view through a viewer, wherein the viewer comprises an obstacle disposed in between an objective lens and a relay lens, wherein the obstacle is to split the initial pattern displayed on the screen as visible to the user, to emulate a principle of diffraction of light;
an input coupled to the screen of the screen, wherein the an input is configured to receive a feedback from the user iteratively and cause the screen to be displaced from an initial position to a final position until the initial pattern is correctly visible to the user;
and a controller configured to detect a refractive parameter of an eye of the user based on a displacement of the screen from the initial position to the final position.

8. The handheld portable autorefractive device as claimed in claim 7, wherein the an input comprises a focus knob coupled to a shaft of an encoder, wherein based on a rotation of the focus knob in a first direction or a second direction encoding signals are generated to cause displacement of the screen.

9. The handheld portable autorefractive device as claimed in claim 7, further comprising a limit switch to restrict a movement of the screen along a longitudinal axis of the screen.

10. The handheld portable autorefractive device as claimed in claim 9, wherein the movement of the screen along the longitudinal axis of the screen lies in a range of +15 to −15 diopters.

11-15. (canceled)

16. A handheld portable autorefractive device comprising:
a light source configured to direct a beam of light towards an eye of a user through a beam splitter arrangement;
a detector, wherein the detector is configured to obtain a reflected beam of light from the beam splitter arrangement, wherein the reflected beam of light is incident on the detector after passing through a micro-array to form a pattern; and
a controller configured to compute a distortion component of the pattern formed on the detector of the detector, and detect a refractive parameter of an eye of the user based on the distortion component.

17. The handheld portable autorefractive device as claimed in claim 16, further comprising:
a viewer, wherein the viewer comprises the beam splitter arrangement, wherein the beam splitter arrangement comprises a first beam splitter disposed in between an objective lens and an obstacle of the viewer along a first axis;
and a second beam splitter disposed substantially perpendicular to the first beam splitter along a second axis.

18. The handheld portable autorefractive device as claimed in claim 16, wherein a shape of a plurality of micro openings of the micro array is any one of a circular shape, an oval shape, a square shape, or a rectangular shape.

19. The handheld portable autorefractive device as claimed in claim 16, wherein the detector comprises a detector lens disposed in between the beam splitter arrangement and the micro array, wherein the detector lens is a singlet lens, a doublet lens, or a combination of a singlet lens and a doublet lens.

20. The handheld portable autorefractive device as claimed in claim 17, wherein the light source is positioned along a first axis and the detector is positioned along a second axis, wherein the first axis and the second axis is substantially perpendicular to one another, such that the first beam splitter, the second beam splitter, and the detector are arranged coaxially.

21. The handheld portable autorefractive device as claimed in claim 17, wherein the detector is positioned along a first axis and the light source is positioned along a second axis, wherein the first axis and the second axis is substantially perpendicular to one another, such that the first beam splitter, the second beam splitter, and the light source are arranged coaxially.
